(19) United States
(12) Patent Application Publication
(10) Pub. No.: US 2012/0183280 A1
(43) Pub. Date: Jul. 19, 2012

(54) FRAGRANCE PRODUCING LIGHTING DEVICE

(75) Inventors: Matthew Gerald Kowalec, Coto De Caza, CA (US); Alan Douglas Rushing, Irvine, CA (US); Brett Robert Moody, Chicago, IL (US)

(73) Assignee: IDC ENCHANTED LIGHTING COMPANY, LLC, Roanoke, TX (US)

(21) Appl. No.: 13/171,730

(22) Filed: Jun. 29, 2011

Related U.S. Application Data
(60) Provisional application No. 61/434,185, filed on Jan. 19, 2011.

(51) Int. Cl. A61L 9/03

(52) U.S. Cl. 392/386

(57) ABSTRACT

An electrically powered device for emitting light and for also producing and releasing fragrances or other vaporizing agents into the surrounding environment through the combined use of a heating element and a polymeric disk that is impregnated with vaporizing agents, at least a portion of which are released into the air upon the application of heat to said disk. In the preferred embodiment of the invention, the heating element and light sources are contained within a base unit that is positioned at a lower portion of the device. The disk, which is formed and shaped so as to optimize exposed surface area, rests on an upper surface of the heating element during operation of the device. The fragrance disk is intended to be removed from the device after a substantial portion of the vaporizing agents impregnated therein are released into the air, allowing for its replacement with another disk.

### CROSS-REFERENCE TO RELATED APPLICATION

This application claims the benefit of U.S. Provisional Application No. 61/434,185, filed Jan. 19, 2011, which is incorporated by reference herein.

### BACKGROUND OF THE INVENTION

1. Technical Field of the Invention

The present invention relates generally to electrically powered lighting devices and in particular, to an electric lighting device capable of dispersing fragrances and other vaporizing agents through the use of a polymeric disk impregnated with said vaporizing agents that are released into the air surrounding the device during operation.

2. Description of the Related Art

Various configurations of electrically-powered fragrance producing lighting devices are known in the prior art. Typically, such prior art devices use various heat sources, such as a tea light or a light bulb, that simultaneously act to emit light and also act to slowly warm wax-based substances that are heavily scented with a fragrance. The heat sources of such devices act to turn the waxy fragrance-scented substance from an initial solid state over time into a liquid state. The typical configuration of such prior art devices requires that a tray containing the waxy substance be placed immediately above the heat/light source so that sufficient heat is applied to said substance. One drawback of the foregoing prior art device configuration is that it generally requires the aforementioned tray containing hot wax to be located at the highest point of the device. Such a configuration can potentially be dangerous to persons and pets and damaging to property if the device or an object on which the device rests, is bumped forcefully enough such that the tray is partially or completely dislodged, allowing hot wax to be spilt out of the tray.

Another drawback to such prior art device configurations is that because the heat source needed to melt the fragrance producing wax or other substance also acts as a light emitting element, said light emitting element is required to be located in very close proximity to the fragrance producing substance so as to sufficiently warm said substance. This requirement that the light/heating source be in close proximity to the fragrance producing substance severely limits available design configurations of such devices, often leading to design compromises that results in less than optimal displays of lighting from the device.

Another drawback of such prior art device configurations utilizing wax type substances and perhaps one of the least desirable aspects of such devices, is the high level of maintenance required of users of such devices. In particular, when the fragrance contained in the waxy substance is exhausted or the user wants to change the substance so that a different fragrance is produced, the wax is usually melted, poured out and the tray containing the substance is cleaned.

The drawbacks mentioned above have not been adequately addressed by the prior art. Therefore, it is the object of the present invention disclosed herein to remedy at least some of the deficiencies found in the prior art and provide other benefits as in further detail below.

### SUMMARY OF THE INVENTION

Accordingly, there is provided herein, in various embodiments, an electrical device that emits light and disperses fragrances or other substances into the air surrounding the device through use of a heating element in conjunction with an interchangeable heat-activated polymer infused with one or more fragrances or other substances. In one aspect of an embodiment of the invention, the lighting device produces light by the use of multiple light emitting diodes (LEDs) capable of producing a flickering flame-like effect. The LEDs are mounted on a base unit that includes circuit components for controlling said light sources to provide the aforementioned flickering effect. The base unit is preferably positioned at a lower portion of the lighting device as compared to prior art configurations of lighting devices discussed above.

In another aspect of the foregoing embodiment, an electrically powered heating element is also mounted on the aforementioned base unit. Additional circuit components within the base unit also control said heating element to provide for optimal temperature ranges. A post positioned in the center of the base unit, the lower portion of which is surrounded by the upper surface of the heating element, supports an array of said LEDs which can be used in a variety of fashions to provide illumination and ambiance.

In a further aspect of the invention, a heat-activated fragrance disk composed of a polymeric material that is impregnated with fragrance vaporizing agents, is the source of the aromas produced by the device. The disk is configured to rest on the upper surface of the heating element and is formed so as to include multiple holes, thus maximizing the surface area of the disk available for exposure to the heat produced by the heating element.

The fragrance disk of the aforesaid embodiment of the invention is intended to be replaced with a new disk after the fragrance impregnated in the disk is exhausted such that it no longer disperses adequate amounts of fragrance into the air during operation of the device. When the fragrance of a disk is exhausted, the entire disposable polymeric fragrance disk can be discarded. This presents an advantage over the prior art in that it is unnecessary to clean any components when a fragrance containing substance is exhausted. This also facilitates easy exchange of fragrances by simply removing the disposable fragrance-infused polymer disk containing one fragrance and replacing it with a different disposable fragrance-infused polymer disk containing an alternative fragrance.

In another aspect of the invention, different embodiments of the lighting device may include various forms of decorative outer housing structures such as a candle shells, ceramic figurines, luminaries, etc. The decorative outer housing structure is formed and positioned so that it at least partially encloses the aforementioned base unit. The outer housing structure is further preferably configured to include one or more openings to allow light from the aforementioned light sources to be emitted to the surrounding environment at whatever intensity is desired. Similarly, openings on the outer housing structure may be formed to provide for venting of both heat from the heating element and aromas from the fragrance disk. Likewise, the decorative housing may be formed and the number and size of the openings chosen so as to create a particular desired ambience when the light sources are activated.

In another aspect of the invention, the fragrance producing lighting device of the present invention may be powered by various means, including, but not limited to, hard-wired A/C power, batteries, and solar power. Moreover, it is contemplated that the lighting and heating aspects of the device may be controlled by the user through the use of hand-operated control units electrically connected to the device or via remote control.

In yet another aspect of the invention, the fragrance producing lighting device of the present invention may include a fan used to aid in dispersal of fragrance from the fragrance disk in conjunction with a heating element or without the use of a heating element. A fan mounted adjacent to the fragrance disk is positioned to force air through venting holes formed in the fragrance disk, thus causing fragrance to be dispersed into the ambient air.

### BRIEF DESCRIPTION OF THE SEVERAL VIEWS OF THE DRAWINGS

A more complete understanding of the invention may be had by reference to the following detailed description when taken in conjunction with the accompanying drawings, wherein:

FIG. 1 shows a perspective and cut-away view of a preferred embodiment of the lighting device of the present invention;

FIG. 2 shows an exploded view of the base unit, light sources, heating element and fragrance disk of the preferred embodiment shown in FIG. 1;

FIG. 3 shows a cross-sectional view of the fragrance disk shown in FIG. 2, along Line 3 therein;

FIG. 4 shows perspective view of an alternate embodiment of the base unit post and fragrance disk of the invention;

FIG. 5 shows a side cut-away view of an alternate embodiment of the lighting device of the present invention;

FIG. 6 shows a side view of a further alternate embodiment of the lighting device of the present invention;

FIG. 7 shows a block diagram representing the electrical power and control circuit used in the preferred embodiment of the present invention; and

FIG. 8 shows a side and cut-away view of an alternate embodiment of the present invention utilizing a fan for fragrance dispersal.

Where used in the various figures of the drawings, the same reference numerals designate the same or similar parts. Furthermore, when the terms “front,” “back,” “first,” “second,” “upper,” “lower,” “height,” “top,” “bottom,” “outer,” “inner,” “width,” “length,” “end,” “side,” “horizontal,” “vertical,” and similar terms are used herein, it should be understood that these terms have reference only to the structure shown in the drawing and are utilized only to facilitate describing the invention.

All figures are drawn for ease of explanation of the basic teachings of the present invention only; the extensions of the figures with respect to number, position, relationship, and dimensions of the parts to form the preferred embodiment will either be explained or will be within the skill of persons of ordinary skill in the art after the following teachings of the present invention have been read and understood. Further, the exact dimensions and dimensional proportions to conform to specific width, length, and similar requirements will likewise be within the skill of the art after the following teachings of the present invention have been read and understood.

Preferred embodiments of the fragrance producing lighting device according to the present invention will now be described in detail with reference to the accompanying drawings.

### DETAILED DESCRIPTION OF THE DRAWINGS

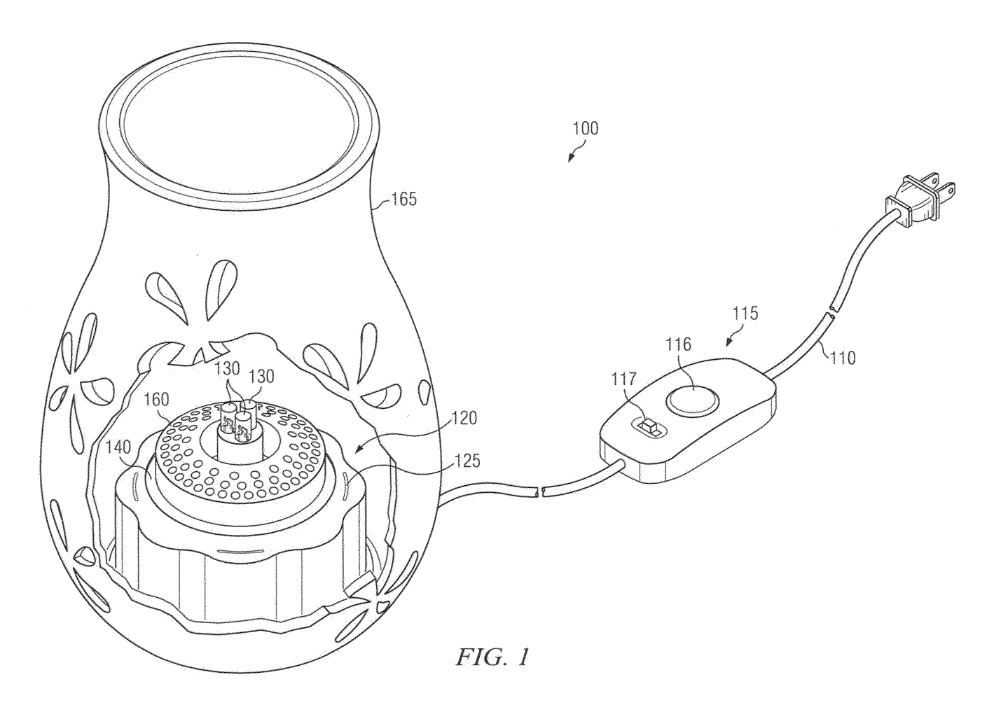

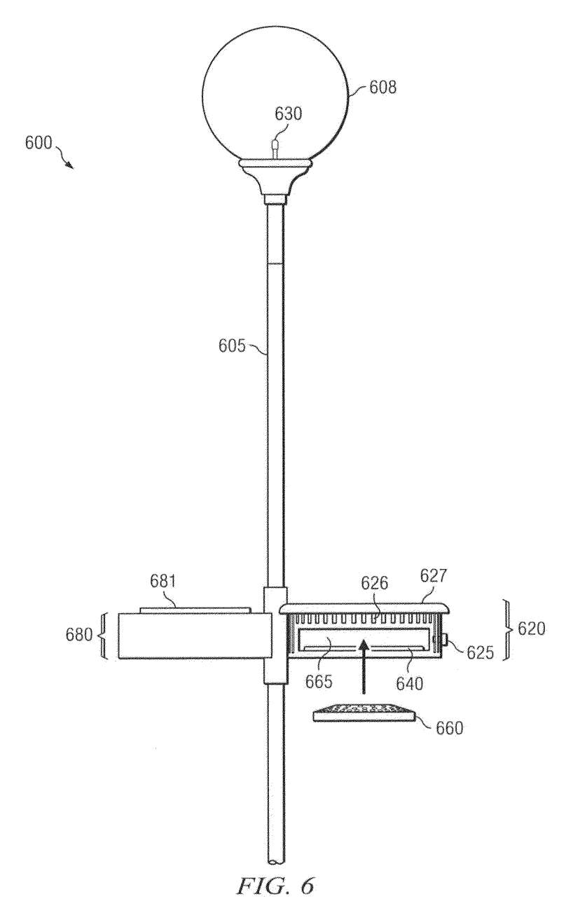

Referring to FIG. 1, a perspective view of a preferred embodiment of the fragrance producing lighting device 100 of the present invention is shown. The lighting device 100 is electrically powered and includes a wired connecting cord 110 providing for connection of the device to an A/C power source such as a common residential 120 volt outlet (not shown). Although the preferred embodiment of the device 100 is configured to be hard-wired to an A/C power source, it is contemplated that alternate embodiments of the invention may be powered by one or more batteries capable of providing the necessary power requirements of the device. Likewise, those of ordinary skill in the art will recognize that other alternate embodiments (such as that which appears in FIG. 6 herein) of the invention intended primarily for outdoor use may include configurations of the device that are powered through the utilization of one or more solar cells and rechargeable batteries and may include a post or stake for supporting the device in the ground or other surface.

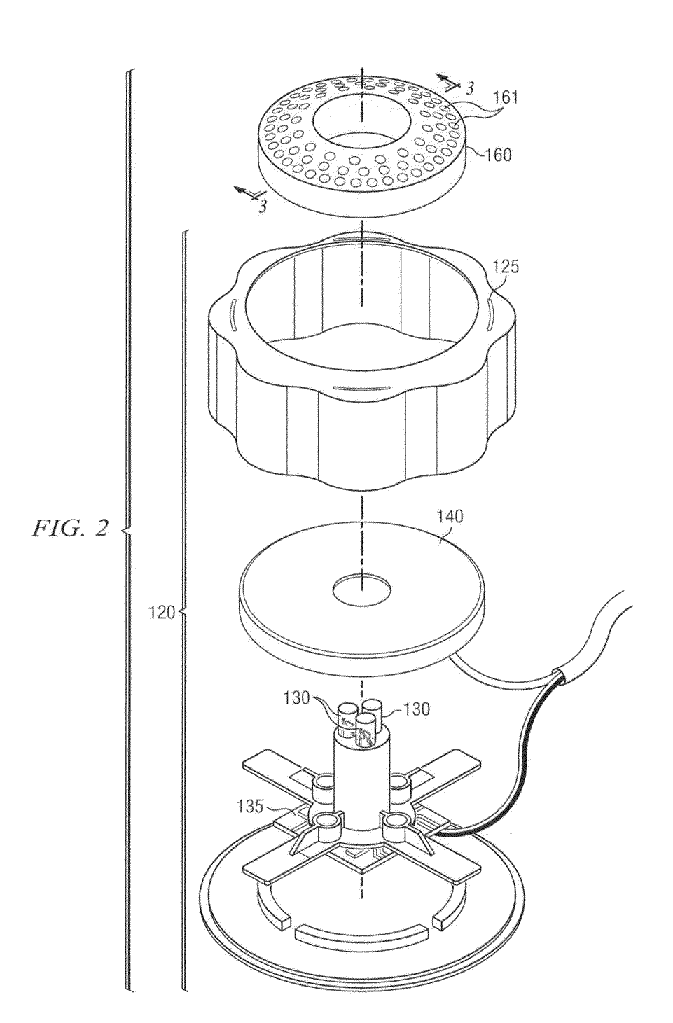

Referring to both FIG. 1 and to FIG. 2, the preferred embodiment of the device includes a base unit 120 that is connected to the connecting cord 110 and a plurality of electric light sources 130 and an electric heating element 140. The housing of the base unit 120, which in the preferred embodiment, is composed primarily of plastic material, serves as a support structure upon which the light sources and heating element may be mounted, as well as a support structure for at least a portion of the circuitry necessary to deliver power to and control the operation of said light sources and heating element. Although not essential to the invention, the housing of the base unit may include slots 125 to allow for venting of heat produced by the heating element. A decorative outer housing 165, discussed in further detail below, surrounds the base unit of the preferred embodiment of the invention and provides for multiple openings to allow for emission of light and dispersal of fragrance.

The heating element 140 of the preferred embodiment includes a heating plate of the type generally known in the art, containing resistive elements (not shown) for generating heat and related control system circuitry (not shown) for controlling the heat produced by said resistive elements. Other heating element configurations capable of producing the heat necessary to optimally operate the device will be known and may be successfully utilized by those of ordinary skill in the art.

It should be noted that it is not essential that the light sources and heating element be located in close proximity to one another in the device. In fact, alternate embodiments of the invention may be configured such that the light source(s) and heating element are located at various distances from one another in the device. The potential for such alternate configurations of the positioning of the light source(s) and the heating element is one advantage over the prior art designs discussed above that require that light source/heating element be the same device component and be in close proximity to the source of the fragrance so that appropriate heat can be transferred.

Referring back solely to FIG. 1, the preferred embodiment includes a control unit 115 that is incorporated into the wired connecting cord 110 so as to provide for user control of the various functions of the device such as activation of the heating element and light sources. In the preferred embodiment, a single push-button power switch 116 is provided to allow a user to control the delivery of power to the light sources. The control unit also includes a three position sliding switch 117 to control delivery of power to the heating element. It should be noted that alternate embodiments of the invention may include a unitary switch controlling both the heating element and the light sources.

Other alternate embodiments of the invention may also include one or more switches or control units integrated into the base unit or outer housing, or the use of a remote control to operate the device. The three position switch 117 within the control unit 115 for controlling the heating element in the preferred embodiment includes switch positions for on, off, and a timer position. A timer control circuit (not shown) allows the heating element to be activated by the user for a pre-determined period of time. In addition to simply being convenient for a user not wanting to unnecessarily waste power, the foregoing timer functionality acts as a safety feature to prevent inadvertently prolonged periods heating element operation.

Light Sources

Referring again to both FIG. 1 and FIG. 2, the preferred embodiment of the invention has multiple light sources and in particular, three light emitting diodes (LEDs) 130. The LEDs 130 are electrically connected to circuitry 135 housed within the base unit, which is in turn electrically connected to a power and control circuit housed in the control unit 115 as shown in FIG. 1. The circuitry includes a printed circuit board (PCB) 135 which is connected to the control unit and the LEDs. Although the preferred embodiment of the invention includes three LEDs, it should be noted that alternate embodiments of the device may include a greater (more than three) or lesser (one or two) number of LEDs or other light sources depending on a number of factors including, but not limited to, the intensity of the light output desired, the power requirements of said light sources, manufacturing costs and the size of the light sources. Moreover, alternate embodiments of the invention may include one or more light sources illuminating at constant intensity.

Fragrance Disk

Still referring to FIG. 1 and FIG. 2, a fragrance disk 160 rests on the upper surface of the heating element during operation of the device. The fragrance disk is the source of the fragrance produced by the device and is replaceable by the user. The fragrance disk of the preferred embodiment is annular in shape, having a circular aperture formed in the central portion of the disk and a plurality of venting holes formed in the annular region of said disk. It should be noted that for the purposes of the teachings and claims herein, the term “disk” shall not be construed so as to be limited to circular or annular shaped forms but may also include any number of various other non-circular polygonal shapes.

In the preferred embodiment, the fragrance disk 160 is at least partially composed of the polymeric material, ethylene vinyl acetate (EVA), a copolymer of ethylene and vinyl acetate that is flexible and remains in a substantially solid state when heated by the heating element. In fact, the EVA disk in its preferred embodiment will not substantially deform at temperatures of less than two hundred degrees Fahrenheit, although some shrinkage of the disk will occur over time as the vaporizing agents are dispersed into the air.

As those with skill in the art will appreciate, the properties of EVA allow it to be impregnated, embedded, or absorbed in or infused with (collectively referred to herein by use of the term “impregnate” or variations thereof) various vaporizing agents such as for example, a fragrance, permitting it to be used as a fragrance reservoir in the device. It should be noted that for the purposes of this specification and the claims recited herein, the term “vaporizing agent” should be construed to mean any substance that may exist in a substantially solid and or liquid state but that is also capable of existing in a substantially vaporized and/or aerosolized state when heated.

Methods for making and using EVA and other polymeric materials and impregnating them with vaporizing agents such as fragrances, odor-neutralizing substances, insecticides and substances having medicinal properties are known in the art. EVA used in the invention may have a molecular weight in the range of 10,000 Daltons to 100,000 Daltons. Vaporizing agents may be impregnated into the EVA at weight percents varying from 10 to 90%, from 20 to 80% from 30 to 70%, from 30 to 60%, and from 30 to 50%. In the preferred embodiment, the fragrance disk is composed of about 75% EVA by weight and about 25% of fragranced vaporizing agent by weight. It should be noted however, that alternate embodiments of the invention may include other combinations by weight of the polymeric material of the fragrance disk and the vaporizing agent.

Alternate embodiments of the fragrance disk may be composed of other polymeric materials including but not limited to, polyethylene (high or low density), polypropylene, polyvinyl chloride, polystyrene, polycarbonate, ABS, PEBAX and polymethylpentene, ethyl vinyl alcohol, polystyrene, acrylic polymers, polycarbonates, polyurethanes, and nylons.

Possible fragrances to be impregnated in the fragrance disk 160 may be selected from the non-exhaustive list of fragrances including musk oil, civet, castretim, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, cauout oil, salvia oil, clove oil, chamomile oil, costus oil, labdanum oil, broom extract, carrot seed extract, jasmine extract, minmosa extract, narcissus extract, olibanum extract, rose extract, acetophenonene, dimethylinadane derivatives, naphthaline derivatives, allyl caprate, alpha-amylcinnamic aldehyde, anethole, anisaldehyde, benzyl acetate, benzyl alcohol, benzyl propionate, borneol, cinnmayl acetate, cinnamyl alcohol, citral citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovaleratek iso-eugenol, linalol, linalyl acetate, p-methylacetophenone, methyl anthranilate, methyl dihydroasmonate, methyl eugenol, methyl-beta-naphthol ketone, methylphenhlcarbinyl acetate, musk ketol, mustk xylol, 2,5,6nanodinol, gannna-nanolactone, phenylacetoaldehydodimethyl acetate, beta-phenylethyl alcohol, 3,3,5trimethylcyclohexanol, gamma-undecalactone, undecenal, vanillin, and mixtures thereof. The foregoing hindered amines may be added to the fragranced vaporizine, agent at weights of between 0.1% and 2.0% by weight of the EVA, preferably at 0.7% by weight. Those of skill in the art will recognize that in constructing the fragrance disk, it is also desirable to mix the fragranced with hindered amines such as for example, 1-(2-hydroxy-2-methylpropoxy)-4-octadecanoyloxy-2, 2,6,6-tetramethylpiperidine; 1-(2-hydroxy-2-methylpropoxy)-4-hydroxy-2,2,6,6-tetramethylpiperidine; and bis(1-oetyloxy-2,2,6,6-tetramethylpiperidin-4-yl)sebacate. Further, various antioxidants such as tertiary butylhydroquinone, butylated hydroxyanisole, phenol bisphosphite, and butylated hydroxytoluene are preferably added to the fragranced vaporizing agent at amounts of between 0.015% and 2.5% by weight of the EVA or other polymer, and preferably between 0.2% and 0.5% by weight.

It is contemplated that in alternate embodiments of the invention, the EVA material used in the disk may also be impregnated with other vaporizing agents such as an insecticide. The insecticide may be selected from the non-exhaustive list of substances including citronella, pyrethrum, methyl bromide, aluminum phosphide, and magnesium phosphide. Other alternate embodiments of the invention may also utilize a disk impregnated with vaporizing agents having medicinal properties. Such vaporizing agents having medicinal properties may be selected from the non-exhaustive list of preparations including eucalyptus oil, menthol, camphor, thymol, turpentine oil, I-desoxyephedrine, and bornyl acetate. In other alternate embodiments of the invention, the foregoing substances may be impregnated in the disk in combination with one or more fragrances or alone. In other words, it is contemplated that alternate embodiments of the disk as disclosed and claimed herein, may or may not be impregnated with fragrances, but only with one or more other vaporizing agents not chosen primarily for the production of a pleasing fragrance.

The beneficial properties of the EVA material used in the disk provide for the distribution of heat necessary to permit an approximately uniform heating effect throughout said disk. When properly heated, the configuration of the disk allows warm air via convection to lift fragrance or other vaporizing agents from the disk into the ambient air. To facilitate this aspect of dispersal, a plurality of smaller (smaller than the central aperture) holes 161 are placed throughout the annular region (region constituting disk) of said disk to accommodate the effect of convection.

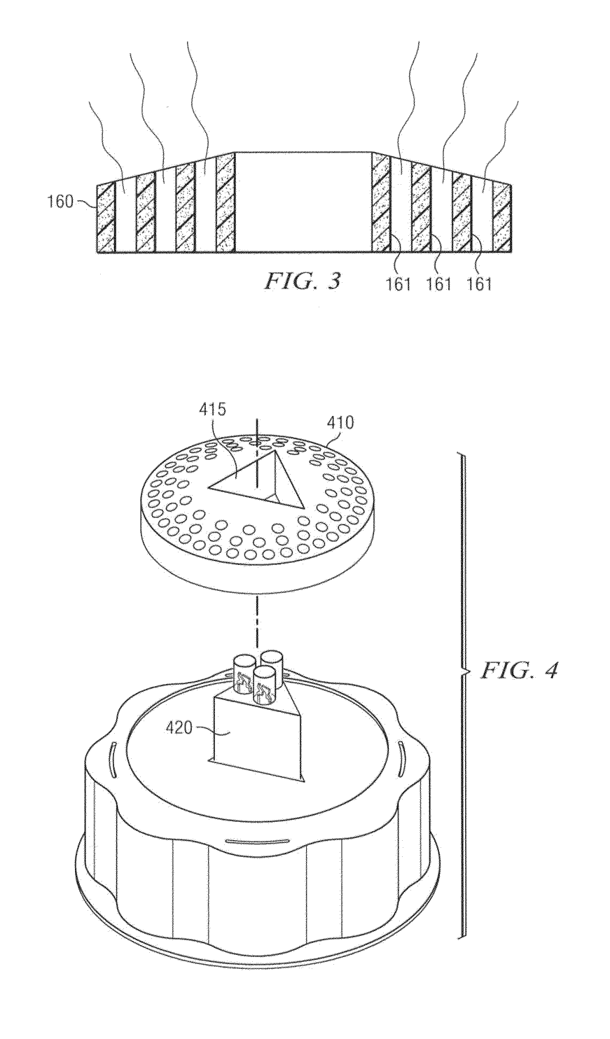

Referring now to FIG. 3, a cross section of the fragrance disk appearing in FIG. 2, a plurality of venting holes 161 are formed in the annular region of the disk. The venting holes 161 are substantially cylindrical columns in form and extend from the bottom of said disk to the top of said disk. It is contemplated that a wide range of shaped venting holes or other openings can be used, including rectangular, cones, slots, etc. Greater numbers of venting holes are preferred as such a configuration results in a higher exposed surface of the disk and thus, increased rates of fragrance (or other vaporizing agent) dispersal into the air.

During operation of the preferred embodiment of the device, the upper surface of the heating element (composed of metal in the preferred embodiment) will preferably have a temperature range of between approximately one hundred, thirty degrees Fahrenheit and one hundred, thirty-five degrees Fahrenheit. As will be recognized by those of skill in the art, other temperature ranges may be used depending on such factors as the density of the polymeric material of the fragrance disk, the properties of the vaporizing agent (fragrances, insecticides, medicinal substances) impregnated into the disk, the desired rate of dispersal of the vaporizing agent, power requirements, and composition and proximity of potentially heat-sensitive components such as the base unit housing and light sources. It should be note that heating of the EVA-composed fragrance disk of the preferred embodiment will cause said disk to reduce in size (referred to as “shrinkage” above) as the impregnated vaporizing agent(s) are dispersed.

Referring to FIG. 1 and FIG. 2, a post is positioned in the center of the annular heating element to support the LEDs. The LEDs are not a significant source of heat and are dedicated to illumination purposes. The post positions the height of the LEDs for desired lighting intensity relative to the specific decorative housing that is being used. In addition to positioning the height of the LED, it is also envisioned that the LED bulbs may also be positionable to direct light as required by the design of the decorative housing. Alternate embodiments of the invention may be configured such that the circuitry controlling the light sources be placed in close proximity to them (for example, at the top of the post supporting the LEDs) or within the heating element housing (for example, below the post).

Referring now to FIG. 4, an alternate embodiment of the fragrance disk 410 and post 420 is shown. Optimal operation of the device of the present invention is likely to occur when the particular composition of the fragrance disk is well-matched with the temperature range of the heating element. If fragrance disks not well suited for use with the heating element of a particular embodiment of the device are used in combination, it is possible that less than desirable results will occur. Accordingly, in alternate embodiments of the lighting device of the invention such as that shown in FIG. 4, posts having outer surfaces with alternative shapes may be used so as to also require the use of fragrance disks having correspondingly shaped central apertures 415. Thus, a user having a fragrance disk intended for use with a particular lighting device and having a central aperture shaped in a manner corresponding to the outer surface of the post of said device, will insert said disk over the post, causing the two elements to mate. Ideally, the foregoing alternate configuration will promote the combined use of fragrance disks intended for use in conjunction with particular lighting devices.

Other Outer Housing Configurations

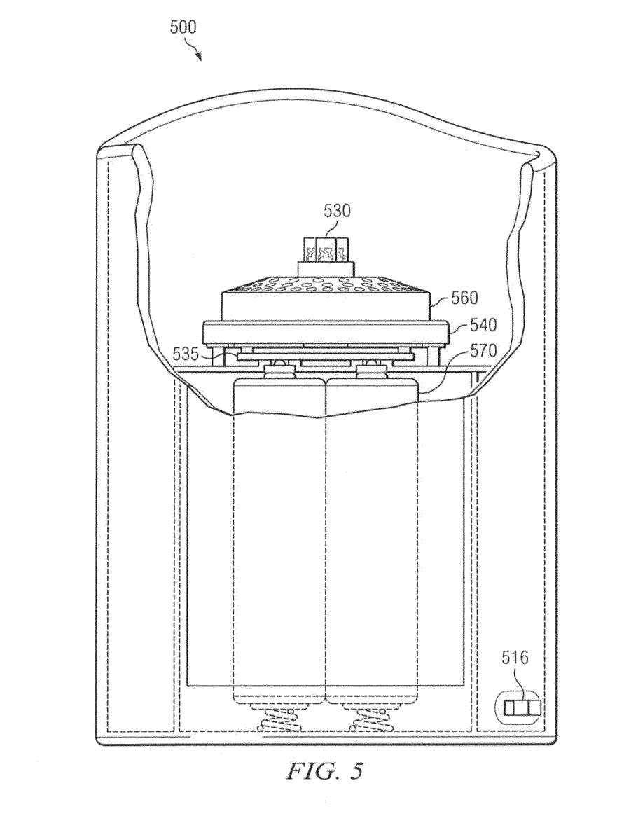

Referring now to FIG. 5, a side cut-away view of an alternate embodiment of the lighting device of the present invention, a candle-like structure serves as a decorative outer housing and support for the base unit and other components of the device. In the alternate embodiment shown at FIG. 5, a cavity is formed in the upper portion of the candle-like housing so as to resemble a genuine candle that has been used. The upper rim of the candle-like housing is also curved to provide the appearance of a genuine candle. Mounted onto the floor of the candle-like housing cavity are the heating element 540, light sources 530, and the power and control circuitry 535 necessary to operate said light sources and said heating element. The fragrance disk used in this alternate embodiment is annular in shape, allowing it to be removably inserted over the light sources and post to rest on the upper surface of the heating element in the same manner as shown in the preferred embodiment appearing at FIG. 1 and FIG. 2.

Still referring to the alternate embodiment of the invention shown at FIG. 5, the device is battery powered. A battery enclosure is formed in the lower portion of the candle-like housing, below the aforementioned floor of the cavity of said housing. Removable batteries 570 mounted within the battery enclosure are electrically connected to the power and control circuitry 535, which is in turn connected to the heating element 540 and light sources 530. It is contemplated that various types of batteries may be utilized to provide power to alternate embodiments of the invention, including but not limited to, alkaline, lithium, nickel-cadmium, nickel-metal hydride, rechargeable, and non-rechargeable batteries. A switch mounted on the candle housing and connected (not shown) to the circuit allows for control of the operation of the heating element and light sources by users of the device. Other user-operated control unit configurations having one or more switches and timers may be integrated into the device or remotely connected to the device in other alternate embodiments of the invention.

Those of ordinary skill in the art will recognize that other various alternate embodiments of the outer housing can be used to enclose and support the other components of the device. For example, luminary type outer housings may be used to provide innumerable decor oriented designs. Such designs may include an opening at the top of the housing, thus providing a unique visual difference in view of prior art fragrance warmers which are configured such that a melting tray is positioned at the top of the device. Materials used to make the decorative outer housing may include wax, plastic, metal, glass, ceramic, and a combination of these or other materials depending on the nature of the decorative housing used.

It may be possible for the decorative housing to attach to the base unit via a variety of methods including a threaded interface (screw-on or twist-on), bayonet lug and slot, or press-fit detents, among others. A shown in the preferred embodiment in FIG. 1, it is also possible that the decorative outer housing may simply removably fit over the base unit with no interface or attaching fasteners needed. The ability to encase the base unit housing in some form of decorative housing improves overall safety of the device as it will isolate the heating portions of the unit to minimize the chance of incidental scalding or burning due to contact with any hot components.

Referring now to FIG. 6 a side view of an alternate embodiment of the lighting device 600 of the present invention is shown. The lighting device 600 of this alternate embodiment takes the term of a solar-powered garden light typically used in outdoor environments. The lighting device includes a tubular body 605 upon which is mounted a glass lens 608. For the purposes of this specification and the claims recited herein, the term “body” should be construed to broadly include any structure upon which the components of the lighting device discussed herein may be mounted. Also for the purposes of this specification and the claims recited herein, the term “lens” should be construed to include any transparent or translucent material through which light may be transmitted.

An LED 630 is mounted on the top of said tubular body 605 and within a cavity formed by a glass lens 608 so as to provide illumination when power is supplied. Mounted on the body is a power unit 680 onto which is mounted a solar cell 681. Rechargeable batteries (not shown) included as part of a power and control circuit adapted for use in conjunction with the solar cell, are mounted within the power unit 680 and connected to the solar cell 681, allowing for the recharging of said batteries when the solar cell 681 is exposed to light. The power unit is electrically connected to the LED 630 and supplies said LED 630 with power stored in the batteries. As is known in the art, the solar cell 681 acts to charge the rechargeable batteries during the daytime when the solar cell is exposed to light. During the nighttime hours, the batteries supply power to the LED 630 to provide illumination. A light sensitive switch (not shown) incorporating a cadmium sulfide (CDS) photoresistor is used to sense ambient light levels such that the aforementioned circuitry causes the LED 630 to illuminate at low light levels.

Still referring to FIG. 6, also mounted onto the body 605 is a base unit 620 which supports a heating element 640 mounted within. The base unit 620 is electrically connected to the power unit 680 such that the rechargeable batteries mounted therein supply the power necessary to operate the heating element 640. Above the heating element 640 is a slot 665 into which a fragrance disk 660 may be inserted by a user such that said fragrance disk may rest on the upper surface of said heating element. A depressible user-operated ejection button 625 is mounted onto the base unit and configured to apply force to the fragrance disk to eject it from the slot 665. In further alternate embodiments, the user-operated ejection button may work in conjunction with a spring-loaded lever to eject the disk. Slots 626 formed on the upper portion of the base unit housing provide for ventilation of fragrances or other vaporizing agents (such as an aerosolized insecticide) into the surrounding air as the fragrance disk is heated by the heating element. An overhang is formed on the top side of the base unit to prevent rain or other water from entering said base unit when the device is placed outdoors. A spike (not shown) is formed on the bottom of the body 605 to facilitate insertion of said body into the ground for outdoor use. Further, one or more switches (not shown) controlling the light sources and/or heating element may be implemented in solar-powered alternate embodiments in the manner discussed herein with respect to the preferred embodiment and other alternate embodiments.

Power and Control Circuit

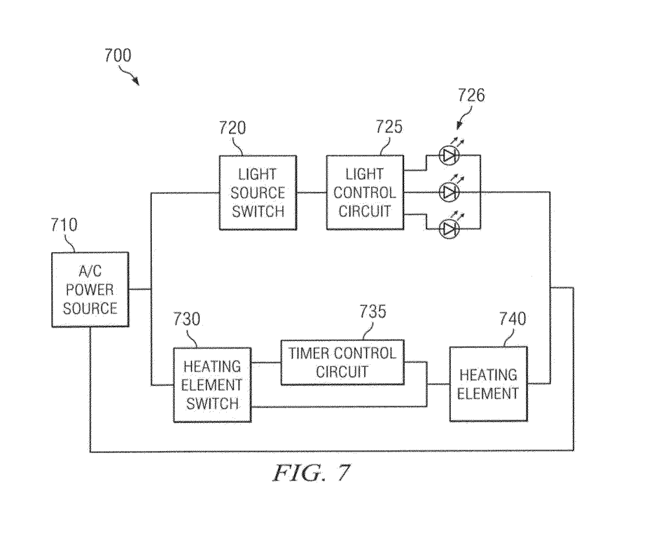

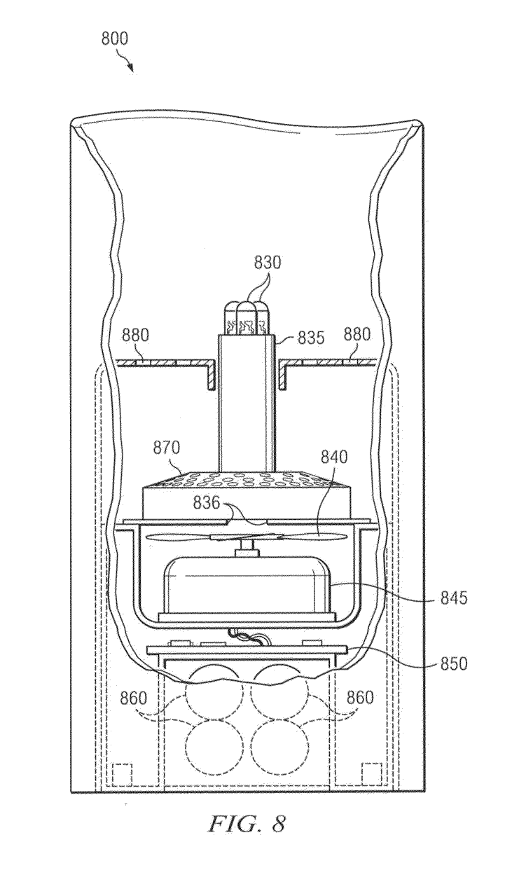

Referring now to FIG. 7, a block diagram representing the electrical power and control circuit 700 used in the preferred embodiment of the present invention is shown. An A/C power source 710 provides the power necessary to operate the light sources 726 and heating element. The A/C power source is connected to the control unit 115 shown in FIG. 1. Within the control unit and also connected to the A/C power source 710 is a light source switch 720 and a heating element switch 730. The light source switch, corresponding to push-button power switch 116 shown in FIG. 1, controls delivery of power to a light control circuit 725 having an integrated circuit and other components for intermittently illuminating the LEDs 726 to produce a flickering flame-like effect.

Various circuit configurations and methods for intermittently illuminating one or more light sources such as LEDs are generally known in the art. The preferred means for producing a flickering light in LEDs utilizes pulse width modulation (PWM) techniques via a controller that sets the duty cycle for each LED. Altering the duty cycles of the current supplied to the LEDs at high frequencies will achieve a flickering flame-like illumination. One example of the use of PWM techniques to produce a flickering effect is taught by U.S. Pat. No. 7,850,327 to Campbell et al., which is incorporated by reference herein. Alternate means for producing a flickering effect may be accomplished by the use of a controller to otherwise adjust how current is supplied to the LEDs. For example, other configurations and methods for delivering power intermittently to light sources to produce a flame-like flickering effect are taught in U.S. Pat. No. 6,719,443 to Gutstein et al., also incorporated by reference herein.

Still referring to both FIG. 7 and FIG. 1, the heating element switch 730 corresponding to the three-position switch 117 shown in FIG. 1, provides for user control of the heating element 740. A timer control circuit 735 is connected to the heating element switch 730 and contains circuitry necessary for activating the heating element for pre-determined periods of time as discussed above. The heating element switch 730 may also be used to directly control power to the heating element without any timing functionality.

In other alternate embodiments of the fragrance producing lighting device of the present invention, methods for activating the heating element and/or a fan intermittently to optimize dispersal of the fragrance or other vaporizing agent may be utilized. Such methods of cycling the power to the heating element and/or fan from an “on” state to an “off” state (and vice-versa) for pre-determined periods of time are generally known in the an as a means for maximizing the dispersal of fragrance and to aid in conserving power.

With respect to the use of such power cycling techniques in connection with a heating element, power is not being actively supplied to the heating element during an “off” state of the cycle and as a result, the upper surface of said heating element cools. Further, during the period in which the heating element is in an “off” state, fragrance that has not yet been dispersed builds up, generally referred to as “head space” in the art. Following the aforesaid “oft” state, the aforementioned power and control circuit acts to again supply power to the heating element, causing said heating element to be in an “on” state and consequently, the upper surface of said heating element to increase in temperature. As the temperature increases, an increased amount of fragrance is dispersed from the disk as a result of the build up of fragrance during the “oft” state of the cycle.

It should be noted that although the time periods of the “on” states and “off” states of the heating element cycle may vary from embodiment to embodiment, the “on” state is preferably set for a period of approximately one minute, followed by an “off” state of approximately four minutes. Similar timing periods may also be employed when the aforementioned power cycling techniques are implemented in connection with embodiments of the device utilizing a fan for the dispersal of fragrance as discussed below.

Referring now to FIG. 8, a side and cut-away view of an alternate embodiment 800 of the present invention, a fan is mounted on a lower portion of a battery powered configuration of the device having a candle-like outer housing structure. LEDs 830 mounted on a post 835 are supported in the upper portion of the housing structure. A fragrance disk rests on support legs of the post 835. Below the fragrance disk is a fan comprising fan blades 840 and a battery powered motor 845 configured to rotate said fan blades. A power and control circuit 850 mounted below the fan motor 845 controls the operation of the LEDs 830 and the fan. During operation of the fan motor 845, the fan blades force air upwards through venting holes (as described above) formed in the fragrance disk, causing fragrance to disperse into the ambient air. Those of ordinary skill in the art will appreciate that although the alternate embodiment of the present invention shown in FIG. 8 does not include a heating element, other alternate embodiments of the invention may utilize a heating element in conjunction with a fan, said heating element being mountable just below the fragrance disk.

The description of the present invention has been presented for purposes of illustration and description, and is not intended to be exhaustive or limited to the invention in the form disclosed. Many modifications and variations will be apparent to those of ordinary skill in the art. The preferred embodiment appearing in the drawings was chosen and described in order to best explain the principles of the invention the practical application, and to enable others of ordinary skill in the art to understand the invention for various embodiments with various modifications as are suited to the particular use contemplated. It will be understood by one of ordinary skill in the art that numerous variations will be possible to the disclosed embodiments without going outside the scope of the invention as disclosed in the claims.

We claim:

1. A fragrance producing lighting device comprising:
(a) an electric power and control circuit connected to a base unit and one or more electric light sources, said electric light sources mounted on said base unit;
(b) an electric heating element mounted on said base unit and electrically connected to said power and control circuit;
(c) a replaceable fragrance disk composed of a polymeric material impregnated with a vaporizing agent; said fragrance disk having a plurality of venting holes formed therein, said fragrance disk adapted for removably mounting on said heating element;
wherein at least a portion of said vaporizing agent is released into the air surrounding said device upon heating of said fragrance disk by said heating element and wherein said fragrance disk remains in a substantially solid state when heated.

2. The fragrance producing lighting device of claim 1, further comprising an outer housing structure adapted to at least partially enclose said base unit, said outer housing structure containing one or more openings.

3. The fragrance producing lighting device of claim 1, wherein each of said one or more electric lighting sources is a light emitting diode (LED), wherein said electric power and control circuit intermittently illuminates at least one of said light emitting diodes independently of said other said lighting sources to provide a flame-like flickering effect.

4. The fragrance producing lighting device of claim 1, wherein said device is powered by an A/C power source.

5. The fragrance producing lighting device of claim 1, wherein said device is powered by batteries.

6. The fragrance producing lighting device of claim 1, wherein said electric power and control circuit includes a control unit connected to a power source and having one or more switches mounted thereto, said one or more switches being connected to said light sources and said heating element to provide for user control of said light sources and said heating element.

7. The fragrance producing lighting device of claim 6, further comprising a timer control circuit connected to at least one of said one or more switches, wherein said timer control circuit is adapted for controlling the time period during which said heating element produces heat.

8. The fragrance producing lighting device of claim 7, wherein at least one of said one or more switches controlling said heating element has three positions comprising an on position, an off position, and a timed position where the heating element will remain activated for a pre-determined length of time.

9. The fragrance producing lighting device of claim 8, wherein said tinier control circuit is adapted to continuously cycle the power to the heating element from an “on” state to an “oft” state for predetermined cycle periods.

10. The fragrance producing lighting device of claim 1, wherein said fragrance disc is at least partially composed of ethylene vinyl acetate (EVA).

11. The fragrance producing lighting device of claim 1, wherein said plurality of venting holes are formed in an annular region of said disk, said holes being in substantially cylindrical shape.

12. The fragrance producing lighting device of claim 1, further comprising a post mounted on a top side of said base unit, said one or more light sources mounted on an upper portion of said post.

13. The fragrance producing lighting device of claim 12, wherein said fragrance disk has a portion thereof defining a central aperture through which said post is inserted upon mounting said fragrance disk to said heating element.

14. The fragrance producing lighting device of claim 13, wherein said central aperture of said fragrance disk is shaped so as to mate with a correspondingly shaped outer surface of said post.

15. The fragrance producing lighting device of claim 1, wherein said vaporizing agent is a substance selected from the group consisting of musk oil, civet, castreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, cauout oil, salvia oil, clove oil, chamomile oil, costus oil, labdanum oil, broom extract, carrot seed extract, jasmine extract, minmosa extract, narcissus extract, olibanum extract, rose extract, acetophenonene, dimethylinadane derivatives, naphthaline derivatives, allyl caprate, alpha-amylcinnamic aldehyde, anethole, anisaldehyde, benzyl acetate, benzyl alcohol, benzyl propionate, borneol, cinnmayl acetate, cinnamyl alcohol, citral citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovaleratek iso-eugenol, linalol, linalyl acetate, p-methylacetophenone, methyl anthranilate, methyl dihydroasmonate, methyl eugenol, methyl-beta-naphthol ketone, methylphenhlcarbinyl acetate, musk ketol, mustk xylol, 2,5,6nanodinol, gannna-nanolactone, phenylacetoaldehydodimethyl acetate, beta-phenylethyl alcohol, 3,3,5trimethylcyclohexanol, gamma-undecalactone, undecenal, and vanillin.

16. A lighting device comprising:
(a) an electric power and control circuit mounted to a body and one or more electric light sources, said electric power and control circuit including a solar cell connected to, and adapted to charge, one or more rechargeable batteries;
(b) an electric heating element mounted onto a base unit that is in turn connected to said body, said heating element electrically connected to said electric power and control circuit, said one or more rechargeable batteries adapted to supply power to said heating element;
(c) a replaceable disk composed of a polymeric material impregnated with a vaporizing agent: said fragrance disk having a plurality of venting holes formed therein; said fragrance disk adapted for removably mounting on said heating element;
wherein at least some of said vaporizing agents are released into the air surrounding said device upon heating of said disk by said heating element and wherein said disk remains in a substantially solid state when heated.

17. The lighting device of claim 16, further comprising a lens forming a cavity through which said one or more electric light sources illuminate light when supplied power from said rechargeable batteries.

18. The lighting device of claim 17, wherein each of said one or more electric light sources is a light emitting diode (LED).

19. The lighting device of claim 16, wherein said vaporizing agent is an insecticide selected from the group consisting of citronella, pyrethrum, methyl bromide, aluminum phosphide, and magnesium phosphide.

20. The lighting device of claim 16, further comprising a slot formed on said base unit, said slot positioned above said heating element and formed to receive said disk.

21. The lighting device of claim 20, further comprising a user-operated ejection button mounted onto said base unit, said ejection button being depressible by the user to apply force to said disk to eject said disk from said slot.

22. A lighting device comprising:
(a) an electric power and control circuit mounted to a device body and one or more electric light sources;
(b) a replaceable disk composed of a polymeric material impregnated with a vaporizing agent; said fragrance disk having a plurality of venting holes formed therein and being mounted within said body;
(c) a fan mounted adjacent to said disk, said fan having a plurality of fan blades and a motor for rotation of said fan blades, said fan being electrically connected to said electric power and control circuit;
wherein when said fan blades are caused to rotate by said motor, air is forced through said venting holes formed in said disk, causing at least some of said vaporizing agents to be dispersed into the air surrounding said device.

23. The lighting device of claim 22, further comprising one or more batteries connected to said electric power and control circuit and supplying power thereto.

24. The lighting device of claim 23, wherein said electric power and control circuit includes a timer control circuit connected to said fan motor, wherein said timer control circuit is adapted for controlling the time period during which said fan motor operates.

25. The fragrance producing lighting device of claim 24, wherein said timer control circuit is adapted to continuously cycle the power to the fan motor from an “on” state to an “off” state for predetermined cycle periods.
